(19) United States
(12) Patent Application Publication
(10) Pub. No.: US 2015/0265753 A1
(43) Pub. Date: Sep. 24, 2015

(54) BREAST PUMP SYSTEM

(71) Applicant: TOMY INTERNATIONAL, INC., Dyersville, IA (US)

(72) Inventors: Ruth Prentice, Cambridge, MA (US); James J. Britto, Westport, MA (US); Patrick Thrailkill, Newton, MA (US); Jeff Tung, Somerville, MA (US); Mike Yoshiaki Daikubara, Quincy, MA (US)

(73) Assignee: TOMY INTERNATIONAL, INC., Dyersville, IA (US)

(21) Appl. No.: 14/432,423

(22) PCT Filed: Oct. 11, 2012
(86) PCT No.: PCT/US2012/059754

§ 371 Date: Mar. 30, 2015

(51) Int. Cl. A61M 1/00; A61M 1/06

(52) U.S. Cl. CPC A61M 1/0031; A61M 1/06; A61M 1/0037

(57) ABSTRACT

A breast pump system. The system includes a pump for generating suction; at least one breast shield; a first bottle operatively coupled to the breast shield for collecting milk; tubing connected between the pump and the breast shield for transmitting suction from the pump to the breast shield to draw milk into the breast shield; and a controller in communication with the pump. The controller is configured to control the pump to generate different suction levels in a cyclic manner at a frequency, control the pump to start and stop pumping, collect input from a user of an amount of milk collected, and store information for at least one pumping session.

### BACKGROUND

The present invention relates to breast pumps and in particular to a breast pump having a controller that stores pumping session information.

Many parents desire to feed their infants, or have their infants fed, with breast milk from the birth mother. Occasionally, a breast-milk provider is unavailable to provide direct breast-feeding to the infant, and must therefore use pre-pumped breast milk stored in a bottle to feed the infant. Though numerous types of breast pumps exist, the easier the pump is for the provider to operate, the more relaxed, and therefore productive, the mother can be. In addition, information about prior pumping sessions helps a nursing mother keep track of trends and optimize milk collection.

### SUMMARY

In one embodiment, the invention provides a breast pump system. The system includes a pump for generating suction; at least one breast shield; a first bottle operatively coupled to the breast shield for collecting milk; tubing connected between the pump and the breast shield for transmitting suction from the pump to the breast shield to draw milk into the breast shield; and a controller in communication with the pump. The controller is configured to control the pump to generate different suction levels in a cyclic manner at a frequency, control the pump to start and stop pumping, collect input from a user of an amount of milk collected, and store information for at least one pumping session.

In another embodiment the invention provides a method of operating a breast pump. The method includes steps of controlling a pump to generate different suction levels in a cyclic manner at a frequency; controlling the pump to start and stop pumping; collecting input from a user of an amount of milk collected during a first pumping session; and storing information for the first pumping session in a controller, the information including the amount of milk collected during the first pumping session. In some embodiments, storing information for the first pumping session in a controller includes steps of measuring an amount of milk collected in a bottle using a scale, communicating the amount of milk measured by the scale to the controller, and storing the amount of milk collected in the controller.

In yet another embodiment the invention provides a graphical user interface on a touch screen display for a controller for a breast pump system. The graphical user interface includes a pump control tab having one or more of a start/stop button, a session duration time clock, a suction control portion, and a speed control portion; a milk volume entry tab having one or more of a left milk volume entry portion, a right milk volume entry portion, a save button, and a session information portion; and a session history tab having one or more of scrolling control portions, and a session history information portion.

Other aspects of the invention will become apparent by consideration of the detailed description and accompanying drawings.

### BRIEF DESCRIPTION OF THE DRAWINGS

FIG. 1 shows a perspective view of an embodiment of the breast pump system of the present invention.

FIG. 2 shows a front view of the touch screen and housing according to an embodiment of the invention.

FIG. 3 shows an exploded view of the housing and associated components according to an embodiment of the invention.

FIG. 4 shows a perspective view of the handle according to an embodiment of the invention.

FIG. 5 shows a pump operation screen according to an embodiment of the invention.

FIG. 6 shows a pump operation screen and session history according to an embodiment of the invention.

FIG. 7 shows a milk volume entry screen according to an embodiment of the invention.

FIG. 8 shows a milk volume entry screen according to an embodiment of the invention.

FIG. 9 shows a session history screen according to an embodiment of the invention.

FIG. 10 shows a setup screen according to an embodiment of the invention.

FIG. 11 shows a diagram of a scale for use with the breast pump system of the present invention.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

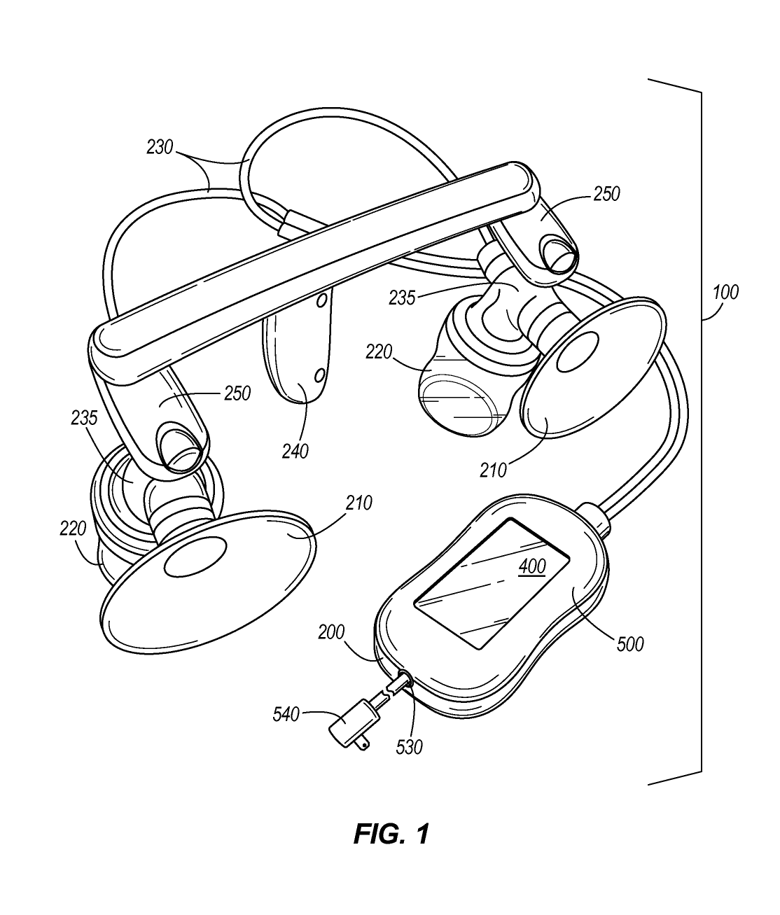

FIG. 1 shows a breast pump system 100 according to an embodiment of the invention. The system 100 includes a pump 200, one or more breast shields 210, a bottle 220 operatively coupled to the breast shield 210 for collecting milk, tubing 230 connected between the pump 200 and the breast shield 210 for transmitting suction from the pump 200 to the breast shield 210 to draw milk into the breast shield 210 and then into the bottle 220, and a controller 300 in communication with the pump 200. In various embodiments, the bottle 220, breast shield 210, and tubing 230 are interfaced with one another using an adapter 235 which provides substantially fluid-tight (e.g. air- and liquid-tight) connections between the various components in order to effectively transfer suction pressure and to collect milk without leakage. In various embodiments the connections between the adapter 235 and the breast shield 210, the bottle 220, and the tubing 230 include suitable gaskets or other sealing mechanisms to ensure substantially fluid-tight connections.

In particular embodiments, the system 100 may include a handle 240 (FIGS. 1, 4) to which two bottles 220 can be attached along with breast shields 210, which in some cases are referred to herein as ‘left’ and ‘right’ bottles 220. In these embodiments, the adapter 235 may also include a connector for attachment to the handle 240. A nursing mother can attach two bottle 220 and breast shield 210 assemblies to the handle 240 (e.g. using the connector on the adapter 235) and thereby nurse from both breasts simultaneously or alternately. The handle 240, which in some embodiments is T-shaped, can also include a pivoting adjustment arm 250 at one or both locations where the bottles 220 attach, where the connector of the adapter 235 couples to the pivoting adjustment arm 250 in a rotatable manner to facilitate proper orientation of the breast shield 210. The adjustment arm(s) 250 allow the user to change the spacing between the bottles 220 to optimize the positions of the bottles 220.

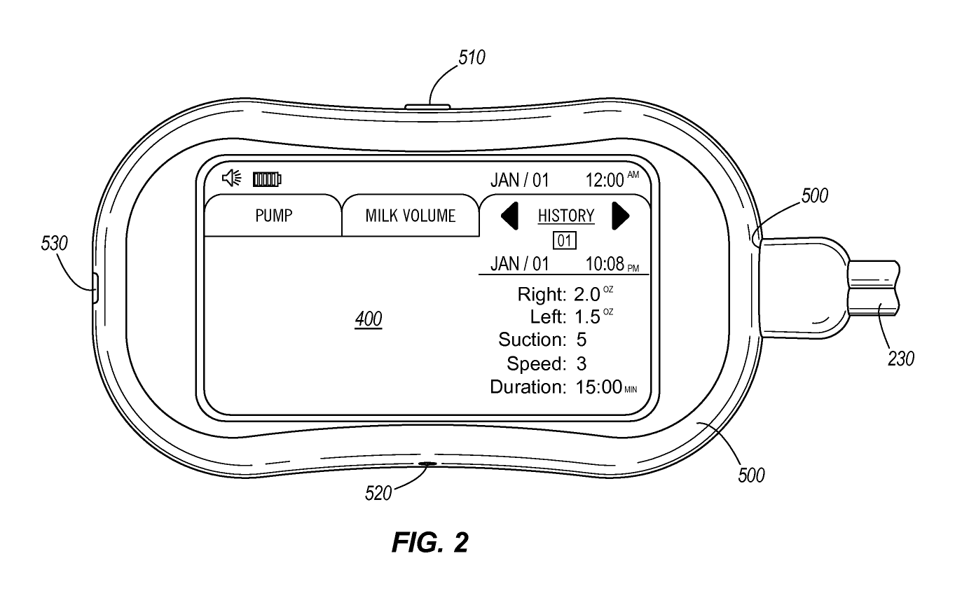

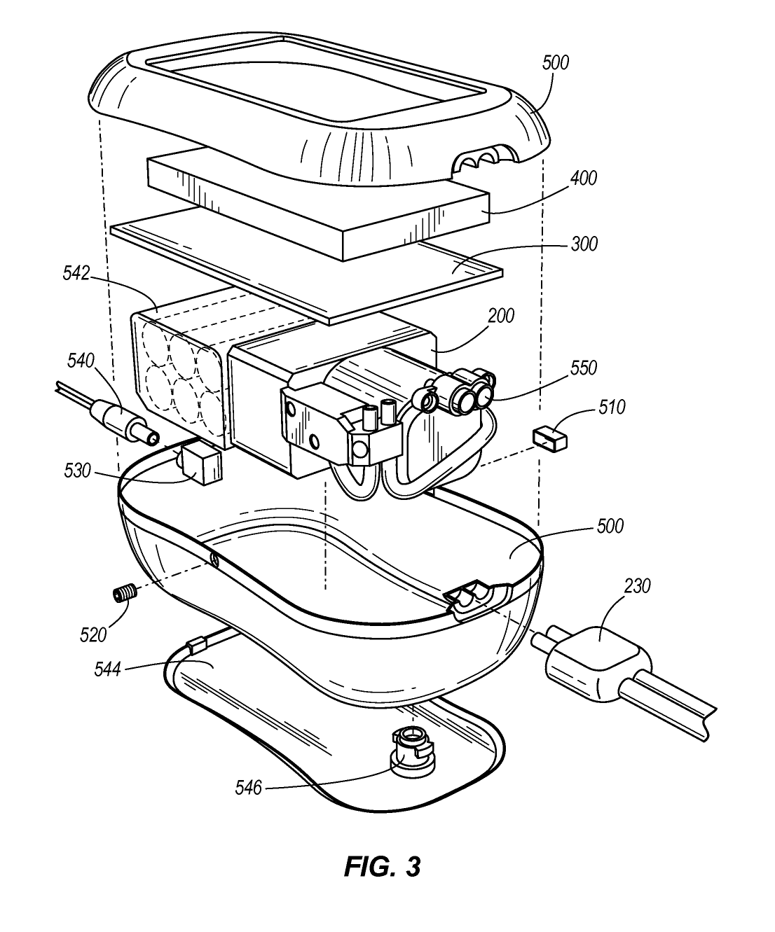

According to embodiments of the invention, the system 100 may include a touch screen 400 in communication with the controller 300 (FIGS. 2-3 and 5-9) and in certain embodiments, the pump 200, controller 300, and touch screen 400 are packaged within a housing 500. In various embodiments, the touch screen 400 includes internal backlighting. FIG. 3 shows an exploded view of the housing 500 along with associated components including the pump 200, a printed circuit board which includes the controller 300, and the touch screen 400. The controller 300 in various embodiments includes a microprocessor and memory and is in communication with the pump 200 and touch screen 400 to facilitate operation of the system 100. As shown in FIG. 3, the housing 500 may include a power button or switch 510, a reset button 520, a receptacle 530 for an external power supply 540, and one or more ports 550 to which the tubing 230 is connected. Pressing the reset button 520 puts the controller 300 into a setup mode, discussed further below.

The external power supply 540 may include a transformer-type plug that converts AC wall outlet power (e.g. 110 or 220 volt AC) to DC power that is then input to the housing 500. The controller 300 and related electronics may operate using the input DC power or may run from an internal battery 542, which is optionally a rechargeable battery. In various embodiments, the internal battery 542 is in a compartment within the housing 500 which is enclosed by a battery compartment cover 544 that is held closed by latch 546.

In operation, a user connects one or two bottles 220 along with respective breast shields 210 to the pump 200 using the adapter 235 and suitable tubing 230, optionally using the handle 240 to hold the one or two bottles 220. The user presses the power button or switch 510 to turn on the controller 300 and touch screen 400 and uses the touch screen 400 to operate the pump 200.

In various embodiments the controller 300 is configured to control the pump 200 to generate different suction levels in a cyclic manner at a particular frequency, start and stop pumping, collect input from a user of an amount of milk collected, and store information for at least one pumping session. Information for at least one pumping session may include one or more of left milk volume, right milk volume, suction level, speed (i.e. cycle frequency), and duration of the session.

During operation, the pump 200 operates in a cyclic manner, alternating between a period of increased suction (e.g. for approximately 0.1-5 seconds) followed by a period of reduced or zero suction (e.g. for approximately 0.1-5 seconds), also called the release phase. Suction pressure is generated during the initial period by operation of the pump 200, whereas the reduced or zero suction of the subsequent period is caused by the opening of a solenoid valve which exposes the tubing 230 to atmospheric pressure.

In some embodiments, there is an additional period between the period of increased suction and the release period. During this additional period, which is referred to herein as a pause, operation of the pump 200 is slowed or stopped (e.g. by reducing or completely eliminating power to the pump 200 which may be accomplished by adjusting a control voltage for the pump 200) for a brief period before the tubing 230 is exposed to atmospheric pressure. The duration of the pause period may vary in length, for example in some embodiments may range between 0.1 and 1.0 seconds, and in one particular embodiment is 0.2 seconds. During the pause, because the pump 200 is generating little or no pressure, the pressure within the system (e.g. within the pump 200, tubing 230, breast shield(s) 210, and/or bottle(s) 220) may increase, stay approximately the same, or decrease. In many cases the pressure may decrease, for example due to small losses of pressure throughout the system. This pause between pumping and release helps to ease the transition between these two phases of the pumping cycle and provides greater comfort for the nursing mother. Thus in some embodiments a complete pumping cycle includes three periods, a first period of increased pressure, a second period in which pumping is paused prior to pressure release, and, finally, a third period in which pressure is released.

The pump 200 repeatedly runs through this cycle for as long as it is turned on. While the pump 200 is operating, the user can increase or decrease the suction pressure using the buttons on the touch screen 400, e.g. using up and down arrows, ‘+’ or ‘−’, or other symbols. In some embodiments the system 100 provides eight different suction levels, which increase the suction during the period of increased suction, whereas the suction level during the period of reduced or zero suction is not affected by this setting. During operation, the user can also increase or decrease the speed of the pump cycle, i.e. the cycle frequency. In various embodiments, the user can select from three different cycle frequencies, where the cycle time (i.e. time to complete the increased suction period plus the reduced or zero suction period) may range from as short as 0.3 seconds up to 11 seconds, and in some embodiments may range from 1 to 6 seconds.

Depending on the mode of operation at a given time, the touch screen 400 may include various portions for accepting input from a user including setting a time of day, setting a date, controlling a volume level, starting the pump, stopping the pump, setting a milk volume, saving session information, reviewing a list of session information, adjusting a suction level, adjusting a cycle speed, a pump tab, a milk volume tab, and a history tab.

Accordingly, in particular embodiments the touch screen 400 includes a graphical user interface for interfacing with the controller 300 of the breast pump system 100. The graphical user interface includes a pump control tab having one or more of a start/stop button, a session duration time clock, a suction control portion, and a speed control portion; a milk volume entry tab having one or more of a left milk volume entry portion, a right milk volume entry portion, a save button, and a session information portion; and a session history tab having one or more of scrolling control portions, and a session history information portion. The tabs and portions displayed in each tab are discussed further below.

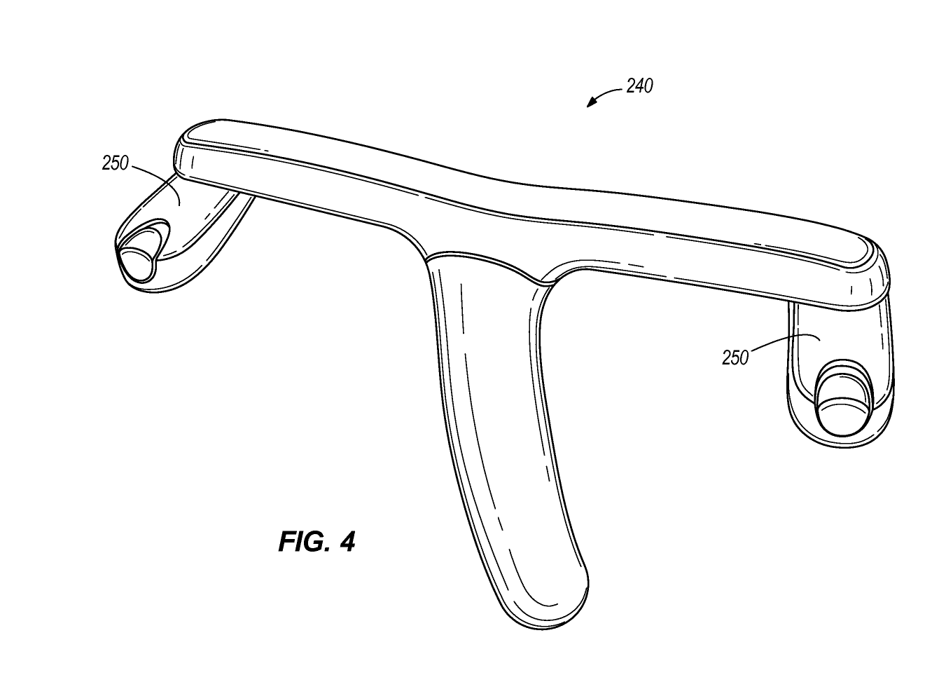

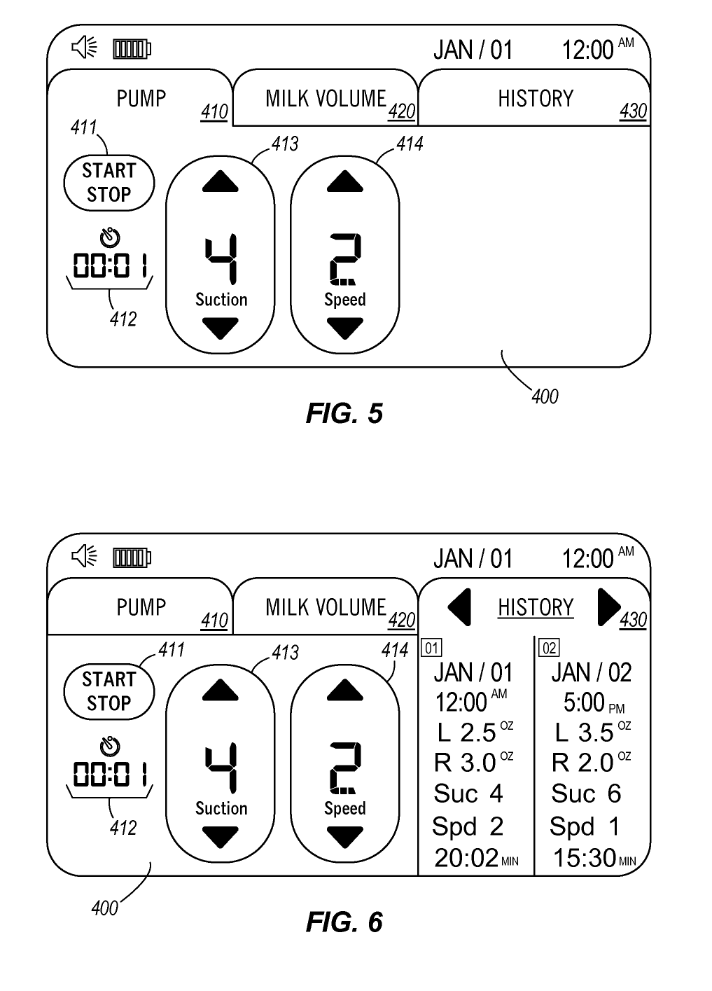

The tabs, which may be displayed across the top of the touch screen 400 or in other suitable locations, provide a way for a user to switch between various modes of operation, e.g. pump control 410, milk volume entry 420, and viewing of session history 430 (FIGS. 5-9). Again depending on the mode of operation at any given time, the touch screen 400 may also include various portions for displaying information to a user including display portions for displaying at least one of session duration time, current pump suction level, current cycle speed level, current volume status (e.g. on or mute), and current battery status. In addition, the touch screen 400 may provide session history information for one or more pumping sessions that have been saved by the user, including one or more of: date, time, left milk volume level, right milk volume level, suction level, speed, and duration.

The user may touch the pump control 410 tab to switch to a pump operation screen (FIG. 5), which includes a start/stop button 411; in some embodiments only the start/stop button 411 is displayed initially. Upon pressing the start/stop button 411, the pump 200 begins operation and a session duration time clock 412 begins running In addition, suction 413 and speed 414 control portions also appear along with suitable up and down arrow buttons (or ‘+’ or ‘−’ or other suitable symbols) to increase and decrease these values and a display to show a current value for each (FIG. 5). When the pump 200 is not running, the start/stop button 411 may display only the word ‘start’ whereas when the pump 200 is running the start/stop button 411 may display only the word ‘stop.’ To begin a session, the user starts the pump 200 by pressing the start/stop button 411 and optionally adjusts the suction and/or speed of the pump 200 using the up and down arrow buttons (or ‘+’ or ‘−’ or other suitable symbols) associated with the suction 413 and speed 414 control portions on the touch screen 400. Upon pressing the start/stop button 411 again and stopping the pump 200, the user has the option of resuming the session (by pressing the start/stop button 411 yet again) or pressing the milk volume entry 420 tab to enter the volume of milk collected in the bottle(s). In some embodiments, the user can press the session history 430 tab (FIG. 6) to display and view the previous session history information while operating the pump 200.

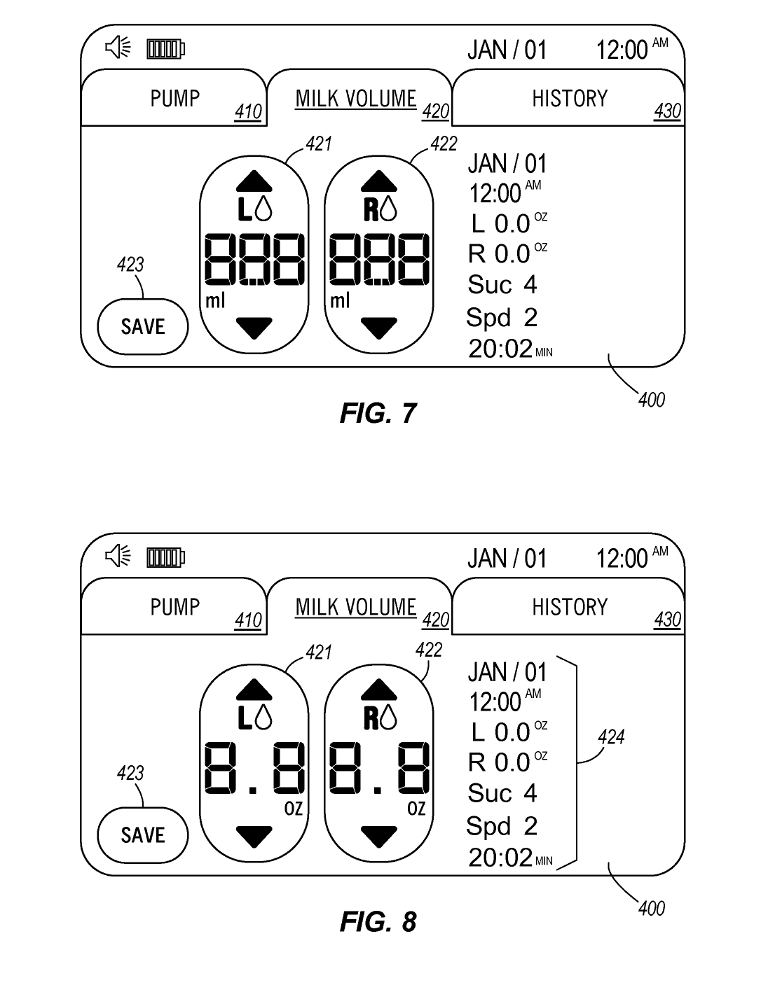

Upon pressing the milk volume entry 420 tab the user can manually enter the volume of milk collected in the bottle(s) 220 during the pumping session (FIGS. 7-8); if no pumping has occurred when the user presses the milk volume entry 420 tab, the touch screen 400 in some embodiments will display a message to the user indicating that milk volume can be entered after pumping. The milk volume entry 420 tab includes left 421 and right 422 entry portions with associated up and down buttons and numerical displays for entering the milk volume information in various increments such as 15 ml (FIG. 7) or 0.5 ounce (FIG. 8) increments. The user can then press a save button 423 on the touch screen 400 to save the session information, which can include duration, right milk volume, left milk volume, suction, and speed. In addition, the milk volume entry 420 tab includes a display of other session information 424.

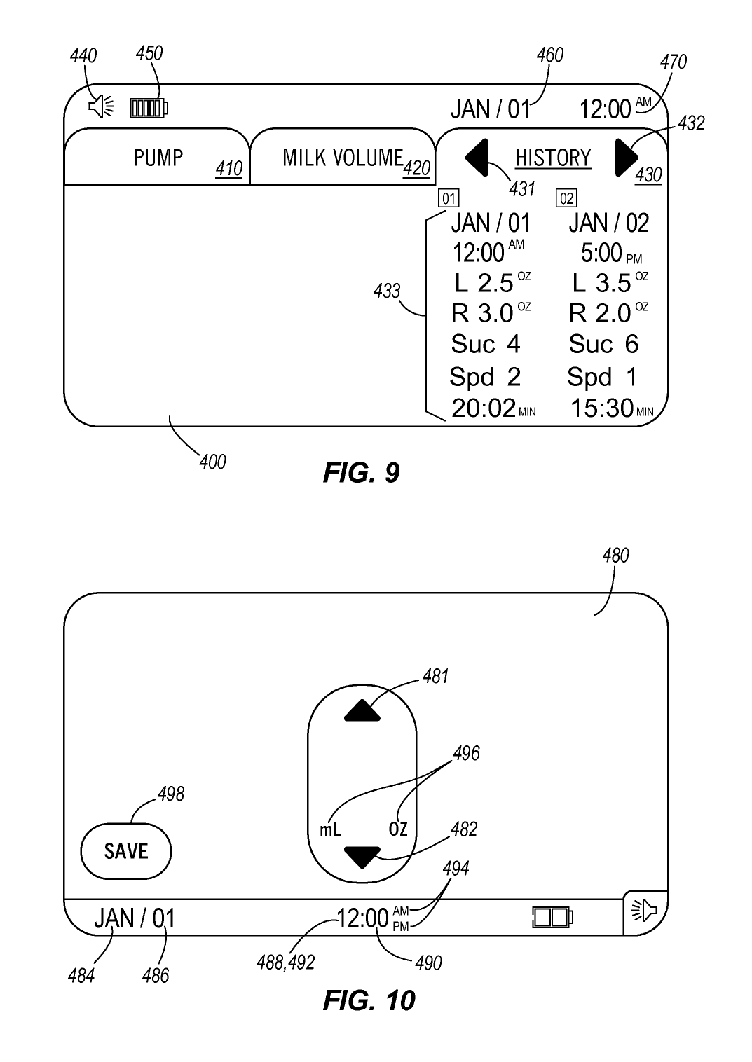

Provided that information for at least one session has been saved, the user can press the session history 430 tab at any time to display session history information, even during a pumping session (FIG. 6). Pressing the session history 430 tab shows information 433 from at least one pumping session and in some embodiments information from two sessions is shown (FIG. 9). The information 433 may include one or more of date, time, left milk volume, right milk volume, suction level, speed (i.e. cycle frequency), and duration of the session (e.g. in minutes and seconds). In addition, the information 433 includes a session number, where in some embodiments information for up to ten sessions may be stored; in other embodiments, information for twenty, fifty, or one hundred sessions or more may be stored, with the number of sessions being stored determined by an amount of memory available. The user can change which session information is displayed using arrow buttons 431, 432 (or ‘+’ or ‘−’ or other suitable symbols).

In various embodiments, the touch screen 400 also includes a volume indicator and control 440, a battery status indicator 450, date 460, and time 470 (FIG. 9), which may be placed in various locations on the touch screen 400. The volume indicator and control 440 shows a suitable icon (e.g. a speaker) from which lines emanate when the volume is on and no lines are shown when the volume is off (muted). The user can toggle between these states by pressing the volume indicator and control 440 icon. In some embodiments a sound (e.g. a beep) is heard when the volume is turned on and similar sounds are emitted each time the user presses a button on the touch screen 400. When the volume is off (muted) no sounds are emitted, although in some embodiments the internal backlighting flashes off and on again each time a touch screen 400 button is pressed when the volume is muted in place of a sound. In some embodiments the internal backlighting flashes off and on again when the volume indicator and control 440 icon is pressed to indicate that the volume has been muted, whereas a sound (e.g. a beep) is emitted when the volume is turned back on. The battery status indicator 450 shows a power level remaining in the battery 542 (e.g. using blocks) and also indicates when the battery 542 is being charged (e.g. by showing one or more blocks flashing on and off).

Pressing the reset button 520 puts the controller 300 into a setup mode (FIG. 10). In setup mode, a user can set parameters including date, time, and a selection of milliliters or ounces for recording milk volume. In some embodiments the user may choose to clear the session history from memory. FIG. 10 shows an embodiment of a setup mode screen 480, in which up 481 and down 482 arrows are used to adjust one or more of a month 484, day 486, hour 488, minute 490, 12-hour or 24-hour clock 492, AM or PM 494, and milliliters (ml) or ounces (oz) 496. In various embodiments, a particular item to be adjusted flashes on the touch screen 400 and the user uses the arrows 481, 482 to adjust a value up or down or to toggle between two or more alternative values for an entry (e.g. AM vs. PM) and then presses a save button 498 to save the entry and to either advance to the next value or to complete the setup if all values have been entered and adjusted. The setup mode can also be implemented using a number of other control symbols such as ‘+’ or ‘−’ or other suitable symbols which properly communicate to a user that the value will be increased or decreased or that an alternate entry will be toggled.

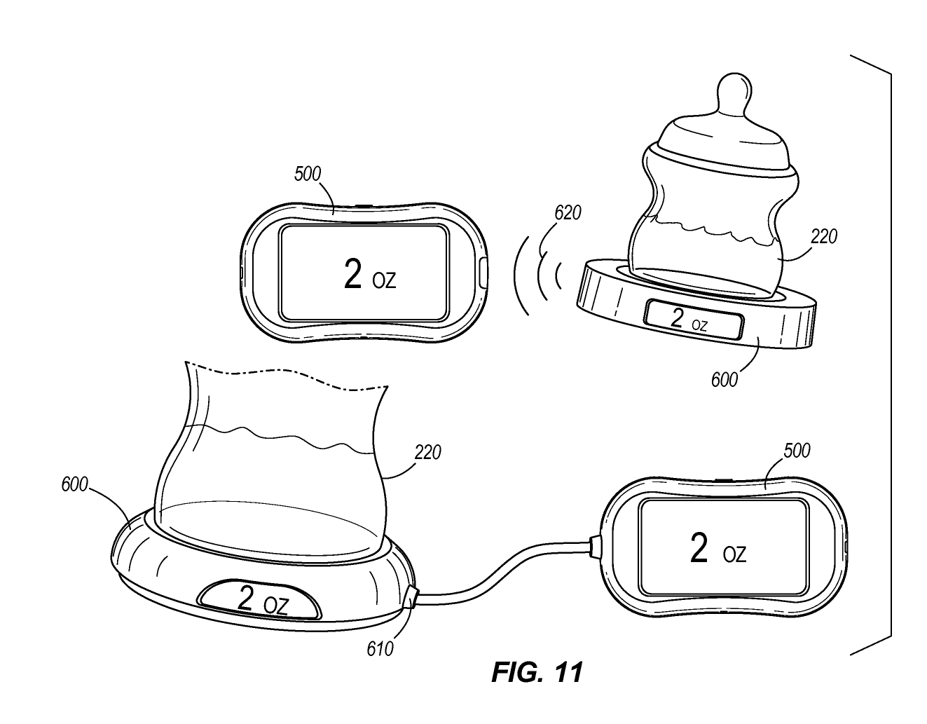

In some embodiments, the system 100 includes one or more accessories such as a scale 600 for automatically determining an amount of milk collected (FIG. 11). In one embodiment, the user first places an empty bottle 220 onto the scale 600 prior to collecting milk and sets the scale 600 to zero (i.e. tares the scale). Once milk collection is complete, the user re-weighs the bottle 220 to determine the weight of the milk. In some embodiments, a standard weight may be deducted from each bottle 220 and in other embodiments the user may store the weight of each bottle 220 in the scale 600, which would then deduct the previously-determined weight of each bottle 220 from the total weight of the bottle 220 plus milk. In the latter embodiment, the scale 600 would store in a memory the weight for up to a predetermined number of bottles 220 (e.g. 5, 10, 20, etc.) with each bottle 220 including an identifier number, with the user then entering the bottle number into the scale 600 when a weight is determined in order to deduct the proper bottle weight.

In some embodiments, the scale 600 reports the amount of milk using liquid units (e.g. milliliters or ounces) and displays the volume of milk to the user. The user may then enter the milk volume using the touch screen 400 controls as discussed above. In other embodiments, the scale 600 automatically transmits the milk volume information to the controller 300, for example using a Universal Serial Bus (USB) connection 610 between the scale 600 and the controller 300 or using a wireless form of communication 620 such as Bluetooth (FIG. 11) or other RF-based signals, infrared (e.g. IrDA), or ultrasonic communications. In various embodiments, the user may be prompted on the touch screen 400 to specify whether the milk volume is for the left or right bottle 220 or a standard procedure may be followed (e.g. left volume always entered first). In some embodiments, the handle 240 may include weighing mechanisms, e.g. a load cell integrated into the adjustment arms 250, which could also communicate in a wired or wireless manner to the controller 300. In other embodiments the scale 600 may be integrated into the housing 500, in which case direct (e.g. wired) communications would be used between the scale 600 and the controller 300.

Although many of the embodiments disclosed herein refer to the touch screen 400 having a limited number of pressure-sensitive pads, in other embodiments the touch screen 400 includes a surface in which the entire area of the screen may be used for receiving input, e.g. using electrical conductivity of a user's fingertip. In such embodiments, alternative control mechanisms may be implemented that are not feasible when using discrete touch pads. For example, a control for a variable (e.g. milk volume) may include a line or bar, with the value being marked by a circle or other item on the line or bar, and the user may adjust the variable by sliding a fingertip across a line or bar to move the circle or the user may touch a particular portion of the line or bar to jump straight to a particular value. In some embodiments the user may select a value (e.g. AM or PM) by pressing a ‘radio button’ to darken a circle and thereby indicate a selection.

In still other embodiments, the breast pump system 100 may be implemented without the touch screen 400 display. Instead, hardwired buttons may be used (some buttons may serve multiple purposes depending on what mode the system 100 is in at a given moment) along with a display that is not touch-sensitive. For example, a single LCD screen or several individual LED or LCD digits and other indicators may be used in conjunction with hardwired buttons to provide the functionality described herein.

Thus, the invention provides, among other things, a breast pump system and a method of operating a breast pump. Various features and advantages of the invention are set forth in the following claims.

1. A breast pump system, comprising:
a pump for generating suction;
at least one breast shield;
a first bottle operatively coupled to the breast shield for collecting milk;
tubing connected between the pump and the breast shield for transmitting suction from the pump to the breast shield to draw milk into the breast shield; and
a controller in communication with the pump, the controller configured to
control the pump to generate different suction levels in a cyclic manner at a frequency,
control the pump to start and stop pumping,
collect input from a user of an amount of milk collected, and
store information for at least one pumping session.

2. The breast pump system of claim 1, further comprising a second bottle and a handle, wherein the first bottle and the second bottle are both coupled to the handle.

3. The breast pump system of claim 2, wherein the first bottle and the second bottle are coupled to the handle using at least one pivoting adjustment arm to permit a distance between the first bottle and the second bottle to be adjusted.

4. The breast pump system of claim 1, wherein information for at least one pumping session comprises at least one of left milk volume, right milk volume, suction level, speed, and duration.

5. The breast pump system of claim 1, wherein the controller, in order to control the pump to generate different suction levels in a cyclic manner at a frequency, is further configured to cyclically operate the pump to increase suction levels, to pause suction pressure generation, and to release suction pressure in a cyclic manner at a frequency.

6. The breast pump system of claim 1, further comprising an adapter that couples the breast shield and the tubing to the first bottle so that milk drawn into the breast shield flows to the first bottle.

7. The breast pump system of claim 1, further comprising a touch screen in communication with the controller.

8. The breast pump system of claim 7, wherein the touch screen includes input portions for inputting at least one of setting a time of day, setting a date, controlling a volume level, starting the pump, stopping the pump, setting a milk volume, saving session information, reviewing session information history, adjusting a suction level, and adjusting a cycle speed.

9. The breast pump system of claim 7, wherein the touch screen includes display portions for displaying at least one of current session duration time, current pump suction level, current cycle speed level, current volume status, current battery status, session history information, current date, current time, and milk volume level.

10. The breast pump system of claim 9, wherein the session history information includes one or more of date, time, left milk volume level, right milk volume level, suction level, speed, and duration.

11. The breast pump system of claim 1, further comprising a scale for determining at least one of a weight and a volume of milk collected in the first bottle, and wherein the scale wirelessly communicates with the controller.

12. (canceled)

13. A method of operating a breast pump, comprising:
controlling a pump to generate different suction levels in a cyclic manner at a frequency;
controlling the pump to start and stop pumping;
collecting input from a user of an amount of milk collected during a first pumping session; and
storing information for the first pumping session in a controller, the information including the amount of milk collected during the first pumping session.

14. The method of claim 13, further comprising storing information for a plurality of pumping sessions and displaying to a user the information for the plurality of pumping sessions.

15. The method of claim 13, further comprising increasing the suction level or increasing the frequency.

16. (canceled)

17. The method of claim 13, wherein collecting input from a user of an amount of milk collected during a first pumping session comprises collecting input from a user of an amount of milk collected during a first pumping session using a touch screen.

18. The method of claim 13, further comprising using a touch screen to collect input from a user including at least one of a time of day, a date, and a volume level.

19. The method of claim 13, further comprising using a touch screen to display information to a user including at least one of session duration time, current pump suction level, current cycle speed level, current volume status, battery status, session history information, current date, current time, and milk volume level.

20. The method of claim 19, wherein the session history information includes one or more of date, time, left milk volume level, right milk volume level, suction level, speed, and duration.

21. The method of claim 13, wherein storing information for the first pumping session in a controller comprises:
measuring an amount of milk collected in a bottle using a scale,
communicating the amount of milk measured by the scale to the controller, and
storing the amount of milk collected in the controller.

22. The method of claim 13, wherein controlling a pump to generate different suction levels in a cyclic manner at a frequency comprises
generating suction pressure for a first period,
pausing suction pressure generation for a second period, and
releasing suction pressure for a third period.

23. The method of claim 22, wherein the second period has a shorter duration than either of the first or the third periods.

24-31. (canceled)
